(19) United States
(12) Patent Application Publication
(10) Pub. No.: US 2018/0361115 A1
(43) Pub. Date: Dec. 20, 2018

(54) DEVICE FOR REMOVING AIR FROM AN ANATOMICAL CAVITY IN A SURGICAL INTERVENTION

(71) Applicant: LUGANO TECHNOLOGY TRANSFER (LTT) SA, Lugano (CH)

(72) Inventors: Stefanos DEMERTZIS, Sorengo (CH); Stijn VANDENBERGHE, Bern (CH)

(73) Assignee: LUGANO TECHNOLOGY TRANSFER (LTT) SA, Lugano (CH)

(21) Appl. No.: 16/060,679

(22) PCT Filed: Jul. 25, 2016
(86) PCT No.: PCT/EP2016/067626

§ 371 Date: Jun. 8, 2018

(30) Foreign Application Priority Data
Dec. 9, 2015 (CH) 1792/15
Dec. 9, 2015 (IT) 102015000081420

(51) Int. Cl. A61M 25/00; A61M 13/00; A61M 25/01

(52) U.S. Cl. CPC A61M 25/0026; A61M 13/003; A61M 25/007; A61M 25/0127; A61M 2210/125; A61M 2025/0037; A61M 2025/0096; A61M 2210/127; A61M 2202/0225

(57) ABSTRACT

A device for removing air from an anatomical cavity in a surgical intervention, comprising a flexible catheter with one or more lumens having a proximal end and a distal end, each lumen having one or more holes at a terminal portion of the catheter comprising the distal end, there being further provided an aspiration means for aspirating air from the anatomical cavity, an insufflation means for insufflating, into the anatomical cavity, an air replacement gas having a higher density than air, and a means for lifting said terminal portion of the catheter for the placement thereof at the top of the anatomical cavity, the insufflation means and said aspiration means being connected to the proximal end of the catheter.

### CROSS-REFERENCE TO RELATED APPLICATIONS

This is a U.S. national phase application under 35 U.S.C. § 371 of International Patent Application No. PCT/EP2016/067626, filed Jul. 25, 2016, and claims benefit of priority to Swiss Patent Application No. 1792/15, filed Dec. 9, 2015 and Italian Patent Application 102015000081420, filed Dec. 9, 2015. The entire contents of these applications are hereby incorporated by reference.

### FIELD OF TECHNOLOGY

The present invention relates to a device for removing air from an anatomical cavity in a surgical intervention.

### BACKGROUND

The preferred, but not exclusive, field of application of the invention is heart surgery. Air is a critical element in surgery, and not only in heart surgery.

Especially when a heart-lung machine is used and an external circuit must be connected to the patient's circulation, air bubbles can enter the patient. Also when anatomical structures full of blood are surgically opened, air can enter the patient's bloodstream.

Moreover, the use of catheters and other hollow instruments can favour the introduction of air into the patient's bloodstream.

Air does not dissolve in the blood but rather creates bubbles of varying diameter which can occlude blood vessels and thus represent a threat to the patient's life if they manage to reach and block cerebral and coronary blood vessels. This can indeed lead to the occurrence of a coronary or cerebral infarction.

Air bubbles of a smaller diameter, though not potentially lethal, can decrease the pumping efficiency of the heart and cognitive functions.

Avoiding the entry of air into the bloodstream is thus a priority in surgery.

When an organ is opened surgically and comes into contact with the atmosphere, it is generally isolated from the patient's blood circulation by clamping the blood vessels.

For the purpose of the intervention it is imperative to assure the complete elimination of the air in the repaired organ before removing the clamps to reconnect the repaired organ to the blood circulation. However, it is not possible to clamp all of the blood vessels in all surgical interventions, so some air can work its way into the bloodstream during the intervention.

The prior art teaches to insufflate CO₂ gas to avoid the accumulation of air in the operating field.

The CO₂ gas is sprayed from a nozzle positioned above the patient and being heavier than air it descends toward the operating field and replaces the air.

The CO₂ gas dissolves in blood and it is highly unlikely that bubbles will form which may jeopardize the patient's safety. CO₂ appears naturally in the blood as a result of metabolism and is expelled through breathing in the lungs.

The prior art in the field of heart surgery also teaches to remove air from the ventricle at the end of a surgical intervention by insufflating CO₂ into the chest cavity and tilting and/or rotating the operating table while air is aspirated from ascending aorta and/or through a suction catheter in the atrium or left ventricle.

CO₂ administered in a traditional manner (insufflation in the operating field) is not, however, always able to reach the most distant anatomical cavities in the heart, which are transformed into air traps.

When the heart resumes beating and injects blood into the ascending aorta, the trapped air reaches the coronary arteries while the suction active in the ascending aorta is in a more distal position and consequently shows limited effectiveness in preventing a coronary embolism.

The technical task of the present invention is thus to provide a device for removing air from an anatomical cavity in a surgical intervention that enables the aforementioned technical drawbacks of the prior art to be overcome.

### SUMMARY

Within the scope of this technical task, one object of the invention is to provide a device that prevents, in an extremely efficacious manner, the introduction of atmospheric air into the operating field during a surgical intervention on an open organ.

Another object of the invention is to provide a device which, after the organ is closed and before it is reconnected to the bloodstream, enables the complete removal of the air which has somehow managed to enter the organ.

Another object of the invention is to provide a device that removes air in a minimally cumbersome manner and without disturbing the surgical field.

The technical task, as well as these and other objects, are achieved according to the present invention by providing a device for removing air from an anatomical cavity in a surgical intervention, characterized in that it comprises a flexible catheter with one or more lumens having a proximal end and a distal end, each lumen having one or more holes at a terminal portion of the catheter comprising the distal end, there being further provided an aspiration means for aspirating air from the anatomical cavity, an insufflation means for insufflating, into the anatomical cavity, an air replacement gas having a higher density than air, and a means for lifting said terminal portion of the catheter for the placement thereof at the top of the anatomical cavity, said insufflation means and said aspiration means being connected to the proximal end of the catheter.

Advantageously, the device simultaneously enables air to be removed in a continuous manner during a surgical intervention and replaced with a replacement gas that is not harmful to the patient.

In the preferred heart surgery application, the catheter is introduced through the aorta so as not to disturb the surgical field.

In a preferred embodiment of the invention, the catheter comprises a first lumen for introducing the replacement gas into the anatomical cavity, which is in mechanical and fluid connection with the insufflation means, and a second lumen for aspirating air from the anatomical cavity, which is in mechanical and fluid connection with the air aspiration means.

In a preferred embodiment of the invention, the insufflation means is configured to deliver a continuous flow of replacement gas into the first lumen.

In a preferred embodiment of the invention, the aspiration means is configured to generate a suction pressure that extracts a continuous flow of air from the second lumen.

In one embodiment of the invention, the lifting means comprises a support wire insertable into one of the lumens of said catheter.

In one embodiment of the invention, said support wire is made of a shape-memory elastic material.

In one embodiment of the invention, said support wire, in a release condition, has two straight portions joined by a bending point which orients said two straight portions transversely to each other.

In another embodiment of the invention, the lifting means is of a magnetic type.

In another embodiment of the invention, the lifting means is of an aerostatic type.

In a preferred embodiment of the invention, the catheter has a means for diverting the flow from the first lumen to the second lumen.

In a preferred embodiment of the invention, the second lumen has a sensor for said replacement gas.

In a preferred embodiment of the invention, the air aspiration means and the insufflation means are connected to a same lumen of the catheter and are selectively activatable in an intermittent manner.

### BRIEF DESCRIPTIONS OF THE DRAWINGS

Additional features and advantages of the invention will become more apparent from the description of preferred, but not exclusive, embodiments of the device for removing air from an anatomical cavity in a surgical intervention according to the invention, illustrated by way of non-limiting example in the appended drawings, in which:

FIG. 1 shows a first embodiment of the device for venting the left ventricle in a mini-invasive mitral valve repair intervention in right-side thoracotomy;

FIG. 2 shows the insertion of the catheter by means of a guide wire;

FIG. 3a shows the catheter positioned in the left ventricle passing through the aorta;

FIG. 3b shows the catheter positioned in the left ventricle passing through the left atrium;

FIG. 4 shows the magnetic-type lifting means of the catheter;

FIG. 5 shows a cross section of the catheter on the lifting means;

FIG. 6 shows a terminal portion of a catheter equipped with a lifting means of a pneumatic type, in a device according to a second preferred embodiment of the invention;

FIG. 7 shows a cross section of the catheter of FIG. 6;

FIG. 8 shows the catheter of FIG. 6 positioned in the left ventricle;

FIG. 9 shows another embodiment of the venting device, in which the lifting means comprises a shape-memory support wire.

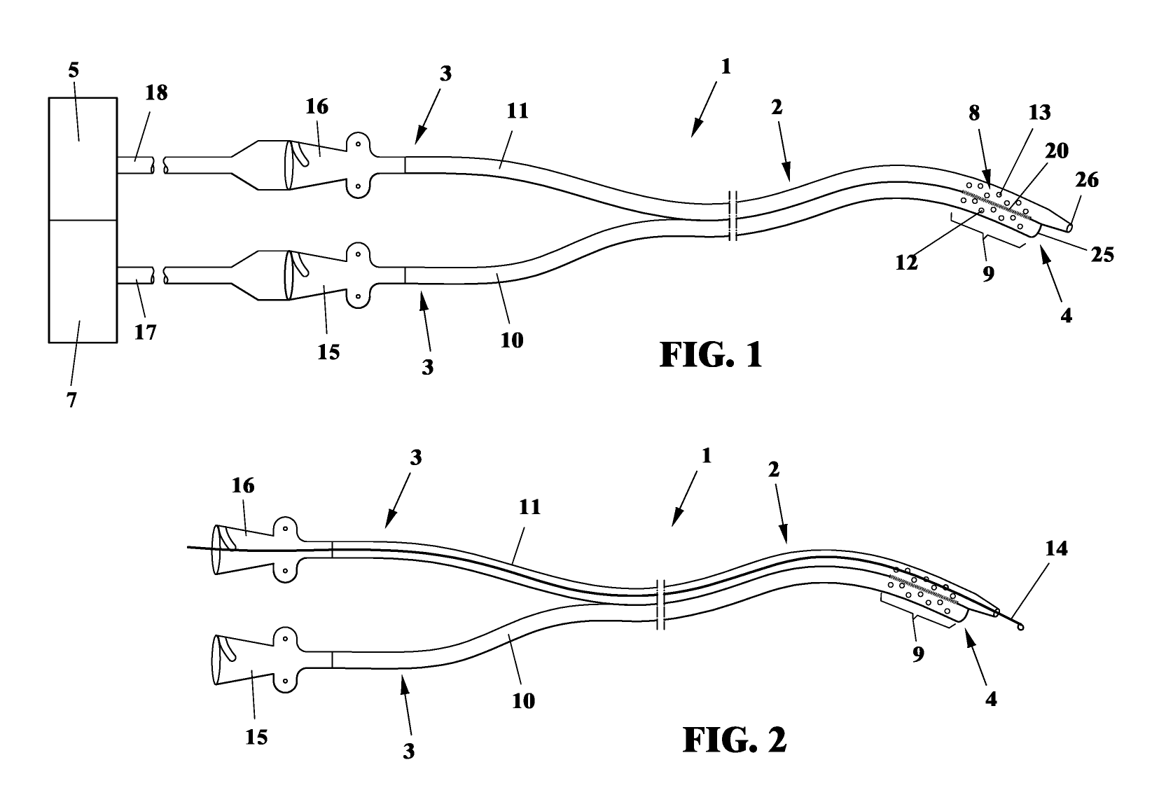

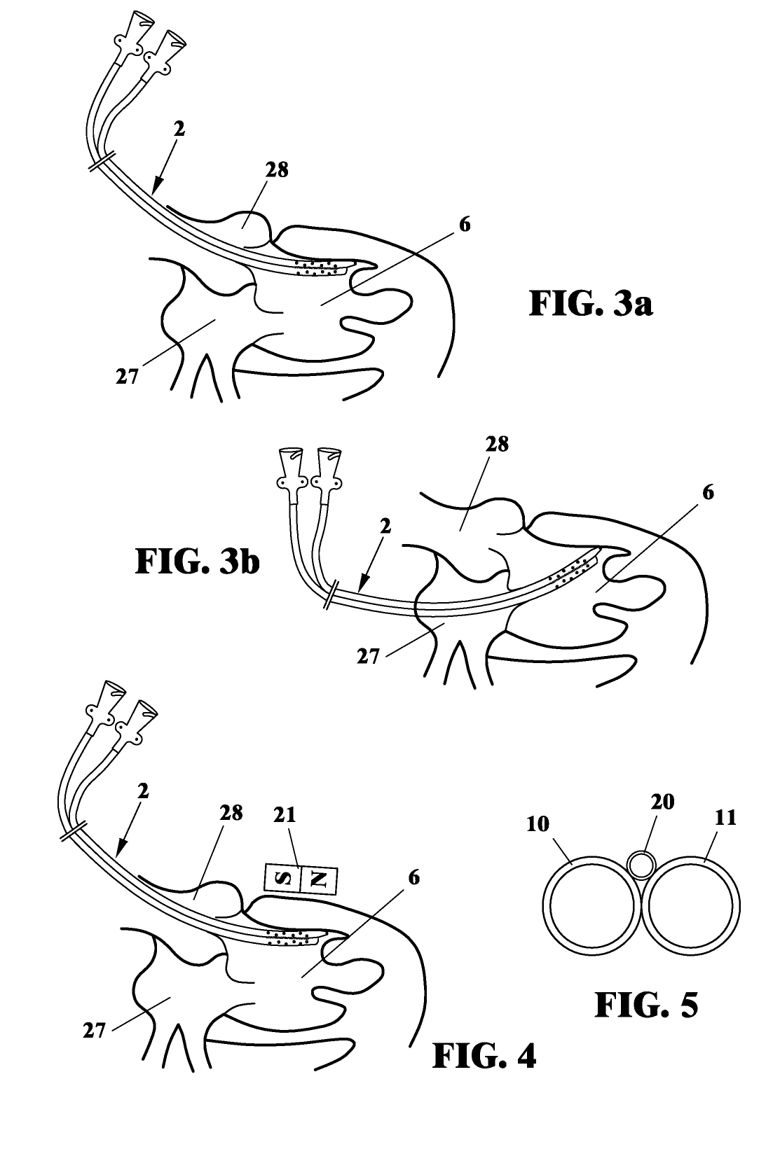

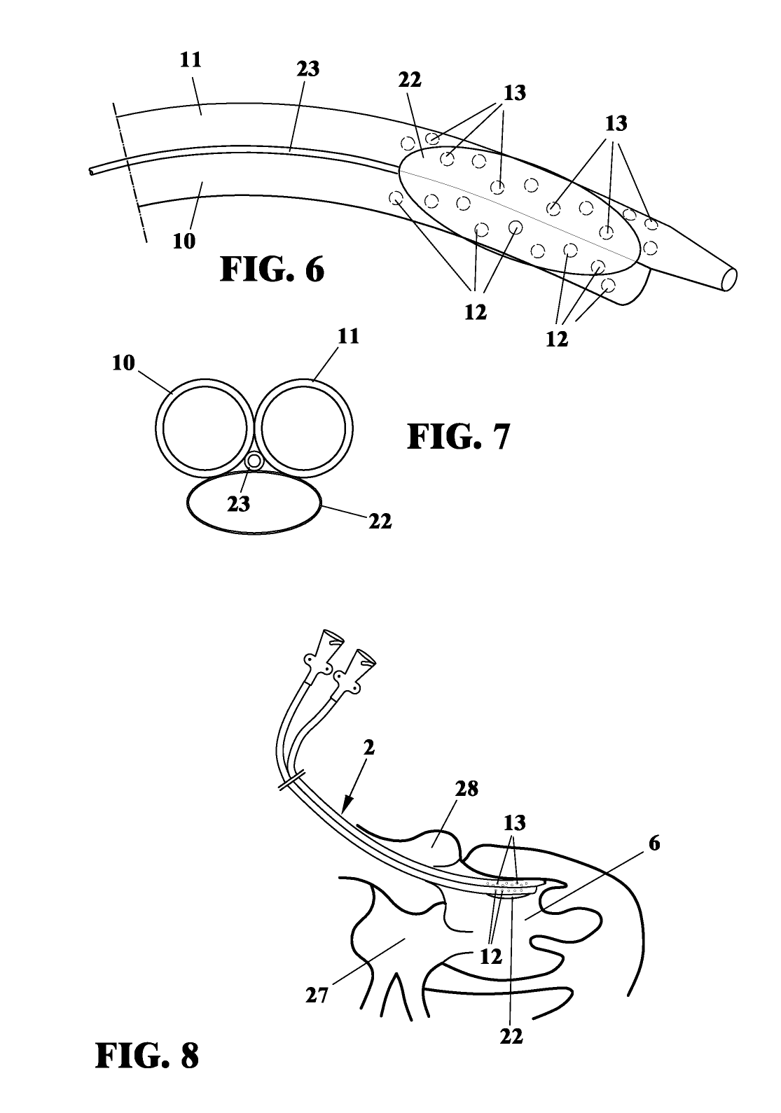

### DETAILED DESCRIPTION

In the description that follows, equivalent parts will be denoted by the same reference number.

With reference to the aforesaid figures, they show a device for removing air from an anatomical cavity in a surgical intervention, denoted in its entirety by the reference number 1.

Hereinafter we will make reference to a venting device in which the anatomical cavity is defined by the left ventricle 6 of the heart, although the scope of application of the device 1 according to the invention extends to other hollow anatomical organs, including blood vessels. In the figures, in addition to the left ventricle 6, the left atrium 27 and the aorta 28 are also shown. The device 1 comprises a flexible catheter 2 with one or more lumens 10, 11 having a proximal end 3 and a distal end 4.

At a terminal portion 9 of the catheter 2 comprising the distal end 4, each lumen 10, 11 has one or more through-holes 12, 13.

The through-holes 12, 13 preferably pass through a terminal portion of the catheter 2 of about 5 cm.

The holes 12, 13 can be circular or oval or of another shape, and their width at the point corresponding to the maximum diameter is about 0.2 mm.

The device 1 further comprises an air aspiration means 5 for aspirating air from the anatomical cavity, an insufflation means 7 for insufflating, into the anatomical cavity of an air replacement gas having a higher density than air, and a means 8 for lifting said terminal portion 9 of the catheter 2 for the placement thereof at the top of the anatomical cavity 6.

The replacement gas is advantageously CO₂.

Both the insufflation means 7 and the aspiration means 5 are connected to the proximal end 3 of the catheter.

More precisely, the catheter 2 comprises a first lumen 10 for introducing the replacement gas into the anatomical cavity 6, which is in mechanical and fluid connection with the insufflation means 7, and a second lumen 11 for aspirating air from the anatomical cavity 6, which is in mechanical and fluid connection with the air aspiration means 5.

The first lumen 10 consists of a flexible tube along which the holes 12 are arranged in succession; the holes can also have a different diameter in order to make the insufflated flow more uniform.

The first lumen 10 further has its proximal end connected, for example by means of a Luer connector 15, to an insufflation tube 17 which is part of the insufflation means 7.

The second lumen 11 consists of a flexible tube along which the holes 13 are arranged in succession; the holes can also have a different diameter in order to make the insufflated flow more uniform.

The second lumen 11 further has its proximal end connected, for example by means of a Luer connector 16, to a suction tube 18 which is part of the aspiration means 5.

The two lumens 10 and 11 are mechanically connected parallel to each other and the holes 12 of one are staggered relative to the holes 13 of the other in the longitudinal direction of the catheter 2.

Preferably, at least the holes 13 of the aspiration lumen 11 are arranged in succession along a path that is not aligned with the longitudinal axis of the catheter 2 in order to prevent the catheter 2 itself from being aspirated against the wall of the anatomical cavity.

The two lumens 10, 11 can differ from each other in shape and/or size and/or in wall thickness, and their cross section can be circular or oval or in any case such as to impart to the catheter 2 a different width and height so that it can be rigid in the direction of height and flexible in the direction of width or vice versa.

The second lumen 11 has its distal end 26 open, whereas the first lumen 10 has its distal end 25 closed and set back relative to the distal end of the second lumen 11.

The opening of at least one of the two lumens 10, 11 is necessary for introducing a guide wire 14 which serves to position the catheter 2 in situ.

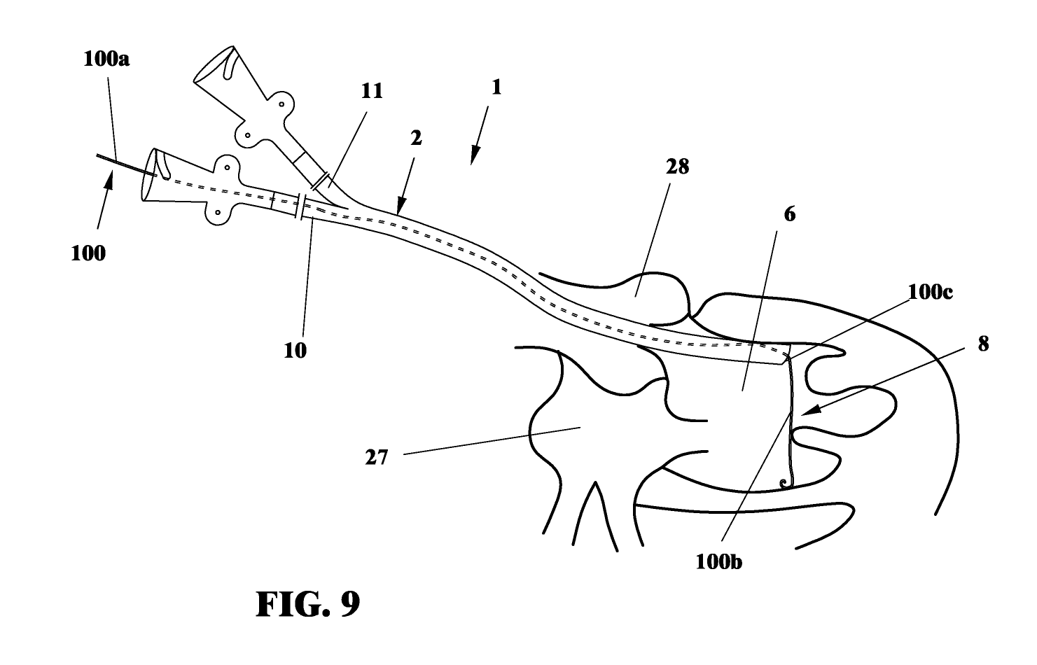

Obviously, both of the distal ends of the two lumens 10, 11 can be open, for example in the embodiment of FIG. 9.

The catheter 2 is made of a material such as polyurethane or silicone or PEBAX or another commonly used one, and the distal end 4 thereof must in any case be made of a soft enough material to avoid damaging the anatomical structures it is made to pass through.

The insufflation means 7 is configured to deliver a continuous flow of replacement gas into the first lumen 10.

The insufflation means 7 can thus comprise a source of replacement gas connected to a feed pump to which the insufflation tube 17 is in turn connected, or else to a pressurized replacement gas tank to which the insufflation tube 17 is in turn connected.

The aspiration means 5, by contrast, is configured to generate a suction pressure that extracts a continuous flow of air from the second lumen 11.

The aspiration means 5 can thus comprise a vacuum pump to the intake of which the suction tube 18 is in turn connected.

The lifting means 8 can be of various types.

In FIG. 9, in the preferred embodiment, the lifting means 8 comprises a support wire 100 insertable into one of the lumens of the catheter 2, in particular into one of the lumens 10, 11 or into another lumen specifically provided for that purpose.

The support wire 100 is made of shape-memory elastic material.

The support wire 100, in a release condition, has two substantially straight portions 100a, 100b joined by a bending point 100c which orients the two straight portions 100a, 100b transversely to each other.

The support wire 100 can be made of a super elastic material such as a nickel-titanium alloy. Typically, the support wire 100 has a circular cross section with a diameter of about 0.5 mm, but it can have a cross section of another shape and size.

The support wire 100 can be pre-shaped by means of a heat treatment and subsequent hardening so that at room temperature, in the release condition, the two straight portions 100a, 100b thereof are joined by the bending point 100c, which defines an angle of about 90°.

Bending of the support wire 100 can be done in one plane (as shown) or also in different planes. The position of the bending point 100c and the bending angle of the support wire 100 can be varied according to need.

The characteristics of the material are such that the bending point 100c can be straightened out in order to insert it into the lumen of the catheter 2 without the support wire 100 undergoing plastic deformation.

To facilitate straightening, at the bending point 100c the support wire 100 can have a different size and/or shape from the remaining part thereof.

The support wire 100 can have a special tip at the distal end, for example a rounded one, to avoid damaging biological tissue.

Moreover, the support wire 100 can have a marker element that indicates the direction of bending and a marker element that indicates when the bending point 100c has come out of the distal end of the catheter 2.

Moreover, the support wire 100 can have a handgrip at the proximal end to facilitate the manoeuvre of insertion thereof.

The positioning of the catheter 2 at the top of the anatomical cavity takes place in the following manner. After the catheter 2 has been inserted into the anatomical cavity by sliding it along a guide wire, and the latter has been removed once the catheter 2 is in the anatomical cavity, the support wire 100 is inserted into a lumen of the catheter 2. The support wire 100 must be straightened by hand or with a specific tool in order to be able to advance along the lumen of the catheter 2. As long as the bending point 100c remains in the lumen of the catheter 2, the support wire 100 will remain straightened due to the containment effect exerted by the wall of the lumen of the catheter 2. When the bending point 100c later comes out of the distal end of the catheter 2 as a result of the advancement of the support wire 100 along the lumen of the catheter 2, the support wire 100 will take on its natural shape again, as there is no longer the constriction of the wall of the lumen. In the absence of constraints on the bending point 100c, the portion 100b will position itself in the anatomical cavity at an angle relative to the portion 100a which remains in the lumen of the catheter 2. In this situation the support wire 100 can be oriented by rotating the portion 100a upon itself in such a way that the distal end of the portion 100b points and pushes against the bottom of the anatomical cavity, thereby acting as pivot that lifts and maintains the catheter 2 positioned at the top of the anatomical cavity.

The lifting means 8 can be either of a magnetic or aerostatic type.

An example of a lifting means 8 of a magnetic type is a magnetic or ferromagnetic element 20, in particular an elastic spiral-shaped metal wire, mechanically connected to the terminal portion 9 of the catheter 2 and suitable for interacting with a magnet 21, in particular an electromagnet or a permanent magnet, which may be appropriately positioned above the operating field.

An example of a lifting means 8 of a magnetic type is a magnetic or ferromagnetic element 20, in particular an elastic spiral-shaped metal wire, mechanically connected to the terminal portion 9 of the catheter 2 and suitable for interacting with a magnet 21, in particular an electromagnet or a permanent magnet, which may be appropriately positioned above the operating field.

An example of a lifting means 8 of an aerostatic type is a balloon 22 that can be inflated with an inflation gas having a lower density than air mechanically connected to the terminal portion 9 of the catheter 2. In this case the catheter 2 is provided with a further lumen 23 for feeding the inflation gas to the balloon 22, and the lumen 23 for feeding the inflation gas is in turn in mechanical and fluid connection with a specific inflation gas insufflation means (not shown), which must be able to reverse its operation to also enable deflation of the balloon when the catheter 2 has to be extracted. The gas insufflation means can comprise a reversible feed pump connected to a source of an inflation gas, which for example is He. The balloon 22 is capable of lifting or at least aiding in lifting the catheter 2, ensuring that it floats on the surface of the saline solution or blood present in the anatomical cavity.

The operation of the device 1, with reference to the application of repairing the mitral valve is briefly as follows. This application will be described, solely by way of non-limiting example, with reference to a lifting means 8 of a magnetic or aerostatic type, given that the operation of the support wire 100 has already been described previously.

The catheter 2 is inserted through the left atrium 28 or through the aorta 27. In the aorta 27, a dedicated puncture site for introducing the catheter 2 can be identified or else the catheter 2 can be introduced through the same puncture site through which the cannula for cardioplegia was introduced.

If the catheter 2 is inserted through the aorta 27, it can be pushed in a retrograde manner toward the aortic valve using a standard technique with the guide wire 14, in which the guide wire 14 is first made to pass through the aortic valve and the catheter 2 is subsequently made to slide on it.

After the catheter 2 arrives in position in the left ventricle 6, the guide wire 14 can be removed.

At this point the lifting means 8 is activated in order to position, and maintain in position, the terminal part 9 of the catheter 2 at the ceiling of the left ventricle 6. Therefore, in the case of the magnetic lifting means 8, the magnet 21 is brought within a range of action surrounding the element 20, whilst in the case of the aerostatic lifting means 8 the balloon 22 is inflated.

At this point the aspiration means 5 and insufflation means 7 are activated.

The insufflation means 7 creates a positive pressure that is maintained constant over time and the aspiration means 5 simultaneously creates a negative pressure that is maintained constant over time.

The filling gas, being heavier than air, comes out of the holes 12 of the first lumen 10 and sinks to the bottom of the left ventricle 6, filling it and replacing the lighter air, which is pushed toward the ceiling of the left ventricle 6, from which it is then aspirated through the holes 13 of the second lumen 11 in order to be finally eliminated.

The CO₂ gas which remains in the left ventricle 6 at the end of the intervention is innocuous and dissolves in the blood.

In this manner, the air is aspirated from the left ventricle 6; however, CO₂ and/or blood can also be aspirated in the absence of air.

Advantageously, there can be provided a diverting means (not shown) capable of intervening in order to interrupt the fluid connection between the aspiration means 5 and the second lumen 11 and establish a fluid connection between the insufflation means 7 and the second lumen 11. The diverting means intervene if and when there is a need to remove obstruction from the second lumen 11. It may occur, in fact, that the second lumen 11 is aspirated against the wall of the anatomical cavity or that blood is aspirated. In this case, the temporary reversal of the flow of gas in the second lumen 11 determines the complete removal of obstruction from the second lumen 11 itself. The diverting means can for example comprise a three-way valve. The diverting means can also be automated in order to intervene every time the suction pressure or the flow of aspirated air in the second lumen 11 falls below a certain threshold value.

Advantageously, the device can be used at the end of the mitral valve repair intervention in order to perform a test on the functionality of the mitral valve itself consisting in rapidly filling the ventricle with an amount of saline solution such as to pressurize the ventricle and induce the closure of the mitral valve.

The device thus conceived is susceptible of numerous modifications and variants falling within the scope of the inventive concept; moreover, all the details are replaceable by technically equivalent elements.

For example, there can be provided a means for adjusting the temperature of the replacement gas and/or inflation gas, which can be heated or cooled within an interval of temperature that is tolerable for the patient in order to optimize the effect obtained by differentiating the density thereof.

Moreover, the second lumen 11 can be equipped with a sensor (not shown) for the replacement gas, based, for example, on the diffraction of infrared light or on transmission with the function of interrupting or temporarily reducing the operating speed of the aspiration means 5 when the gas replacement sensor detects only CO₂, without air, in the flow.

Finally, a simplified version of the device according to the invention envisages that the air aspiration means 5 and the insufflation means 7 are connected to a same lumen of the catheter 2 and can be selectively activated in an intermittent manner. In practical terms, an automated valve system switches over to connect, in a selective and alternating manner, the insufflation means 7 and the aspiration means 5 to a same lumen of the catheter 2. In this case an alternating sequence of insufflations of filling gas and aspirations of air from the anatomical cavity occurs.

1. A device for removing air from an anatomical cavity in a surgical intervention, comprising:
a flexible catheter with one or more lumens having a proximal end and a distal end, each lumen having one or more holes at a terminal portion of the catheter comprising the distal end,
an aspiration device aspirating air from the anatomical cavity,
an insufflation device insufflating, into the anatomical cavity,
an air replacement gas having a higher density than air, and
a lifter lifting said terminal portion of the catheter for the placement thereof at the top of the anatomical cavity,
wherein said insufflation device and said aspiration device being connected to the proximal end of the catheter.

2. The device for removing air from an anatomical cavity according to claim 1, wherein said catheter comprises a first lumen introducing the replacement gas into the anatomical cavity, which is in mechanical and fluid connection with the insufflation device, and a second lumen aspirating air from the anatomical cavity, which is in mechanical and fluid connection with the aspiration device.

3. The device for removing air from an anatomical cavity according to claim 2, wherein said insufflation device is configured to deliver a continuous flow of replacement gas into the first lumen.

4. The device for removing air from an anatomical cavity according to claim 2, wherein said aspiration device is configured to generate a suction pressure that extracts a continuous flow of air from the second lumen.

5. The device for removing air from an anatomical cavity according to claim 2, wherein said first and second lumens are mechanically connected in parallel to each other.

6. The device for removing air from an anatomical cavity according to claim 1, wherein said replacement gas is CO₂.

7. The device for removing air from an anatomical cavity according to claim 1, wherein said lifter comprises a support wire insertable in one of the lumens of said catheter.

8. The device for removing air from an anatomical cavity according to claim 7, wherein said support wire is made of shape-memory elastic material.

9. The device for removing air from an anatomical cavity according to claim 8, wherein q0 said support wire, in the release condition, has two straight portions joined by a bending point which orients said two straight portions transversely to each other.

10. The device for removing air from an anatomical cavity according to claim 1, wherein said lifter is magnetic.

11. The device for removing air from an anatomical cavity according to claim 1, wherein said lifter is aerostatic.

12. The device for removing air from an anatomical cavity according to claim 1, further comprising an adjustor adjusting the temperature of the replacement gas.

13. The device for removing air from an anatomical cavity according to claim 2, further comprising a flow diverter diverting the flow from the first lumen to the second lumen so as to remove obstruction from the latter.

14. The device for removing air from an anatomical cavity according to claim 2, characterized in that said second lumen has a sensor for said replacement gas.

15. The device for removing air from an anatomical cavity according to claim 1, characterized in that said air aspiration means and said insufflation means are connected to a same lumen of the catheter and are selectively activatable in an intermittent manner.
